(19) United States
(12) Patent Application Publication
(10) Pub. No.: US 2017/0056367 A1
(43) Pub. Date: Mar. 2, 2017

(54) PHARMACEUTICAL COMPOSITIONS FOR INHIBITING THE OSTEOCLAST DIFFERENTIATION COMPRISING EUPATILIN

(71) Applicant: OsteoNeuroGen CO., LTD, Gyeonggi-do (KR)

(72) Inventors: Byung Soo YOUN, Seoul (KR); Jae Min OH, Jeollabuk-do (KR); Ju Young KIM, Jeollabuk-do (KR); Myeung Su LEE, Jeollabuk-do (KR)

(21) Appl. No.: 14/834,441

(22) Filed: Aug. 25, 2015

(51) Int. Cl. A61K 31/352

(52) U.S. Cl. CPC A61K 31/352

(57) ABSTRACT

Disclosed is a pharmaceutical composition comprising eupatilin as an active ingredient for suppressing osteoclast differentiation, bone metastasis, and osteolysis and for preventing or treating periodontitis. Found to effectively inhibit the differentiation of osteoclast progenitors into osteoclasts and the fusion of mononucleated osteoclasts into multinucleated osteoclasts, and reduce the bone resorption of W multinucleated osteoclasts, Eupatilin is capable of effectively suppressing bone metastasis and osteolysis and may be used to prevent or treat periodontitis.

### BACKGROUND OF THE INVENTION

1. Field of the Invention

The present invention relates to a pharmaceutical composition for use in the inhibition of osteoclast differentiation, bone metastasis, and osteolysis, and the therapy and prophylaxis of periodontitis. More particularly, the present invention relates to a pharmaceutical composition comprising eupatilin as an active ingredient for the inhibition of osteoclast differentiation, thereby inhibiting bone metastasis and osteolysis and preventing or treating periodontitis.

2. Description of the Related Art

Bone is constantly being created and replaced in a continual process of destruction or resorption of bone tissues and the formation of new bone. In this context, osteoclasts function to destroy or resorb bone tissues while osteoblasts are responsible for ossification. Good balance between osteoclasts and osteoblasts is required for the maintenance of healthy bones. For postmenopausal women and the elderly, various factors involved in maintaining the balance become interrupted to induce the excessive differentiation of osteoclasts. Accordingly, the resorption of bone tissues is accelerated, causing various diseases.

Representative among the diseases caused by the excessive differentiation of osteoclasts are osteoporosis and periodontitis. After menopause, a decrease in the production of the female hormone estrogen accelerates the differentiation of osteoclasts and decreases bone density, resulting in osteoporosis. This mechanism is highly reproducible in vivo and in vitro. Under an inflammatory condition such as periodontitis, the differentiation of osteoclasts is also likely to be promoted, thereby increasing bone loss.

Mature osteoclasts are multinucleated giant cells resulting from the fusion of osteoclast progenitors, with an actin ring tying the cells. Osteoclast progenitors are of myeloid cell lineage. When treated with M-CSF (macrophage-colony stimulating factor), myeloid cells are differentiated into preosteoclasts and macrophages. The RANK-ligand (RANKL), known as a master regulator for osteoclast differentiation, binds to the receptor RANK on osteoclasts to trigger various osteoclastic (osteoclastogenic) activities. During osteoclastogenesis, RANKL induces the activation of Ca²⁺-dependent kinase through calcium oscillation, triggering the activation of the transcription factor NFAT-c1, with the resultant expression of various proteins necessary for the differentiation of osteoclasts. In response to RANKL signaling, in addition, cell communication (coupling) factors that regulate the expression level and rate of osteoclasts after expression in osteoblasts or osteoclasts, such as TGF-β, IGF, IFN-γ, TNF-α and SemaD, are involved in the differentiation and apoptosis of osteoclasts in homeostasis and diseased states.

Artemisia is a large, diverse genus of plants with between about 400 species belonging to the compositae family. Of them, 300 species are estimated to grow in Korea, but in fact, only 40 species have been reported thus far. Artemisia is be classified into those that can be used as food only, medicinal herbs only, and both. Artemisia is rich in chloroplasts with various enzymes and nucleic acids, and contains fibroid materials, proteins, minerals, and various vitamins. In addition, Artemisia is a strong alkaline food that can be healthful for an acidity-biased body constitution and can clean oily blood. In herbal medicine, it is also used as a medicament that can prevent disharmony between yin and yang qi in the body and which is helpful for blood circulation and digestion and effective for removing noxious materials. Rich in vitamins A and C, Artemisia can function to increase immunity and aid to prevent the body from catching a cold. Effective components found in Artemisia include isocoumarin, coumarin, diterpene lactone, flavonoid, phellandrene, couprol, cadinene, cineol, artemisinin, and eupatilin. Of them, artemisinin is used as a therapeutic agent for malaria. Recent research has revealed the therapeutic effectiveness of artemisinin on cancer.

Pharmaceutically, a water extract of Artemisia inhibits plasma coagulation and the flavones from Artemisia are demonstrated to show anticancer effects. Having antibacterial and anti-oxidative functions, in addition, the extracts are effectively applied to the treatment of allergies or inflammation. Eupatilin, also termed 5,7-dihydroxy-3,4,6-trimethoxyflavone, has an empirical formula of C₁₈H₁₆O₇ with a molecular weight of 344.3 and a melting point of 232˜233° C.

Leading to the present invention, intensive and thorough research into the development of a natural novel medicinal agent for the prevention or treatment of diseases or disorders caused by the excessive differentiation of osteoclasts resulted in the finding that eupatilin, present in Artemisia, can effectively inhibit osteoclast differentiation and can thus be useful in preventing and treating diseases induced by excessive osteoclast differentiation, such as osteoporosis, periodontitis, and osteolysis.

### RELATED ART DOCUMENT

[Patent Document]

Korean Patent No. 10-0718490

### SUMMARY OF THE INVENTION

It is a primary object of the present invention to provide a pharmaceutical composition that is effectively inhibitory of osteoclast differentiation.

It is another object of the present invention to provide a pharmaceutical composition for the prophylaxis or therapy of diseases or disorders caused by excessive osteoclast W differentiation.

### BRIEF DESCRIPTION OF THE DRAWINGS

The above and other objects, features and advantages of the present invention will be more clearly understood from the following detailed description taken in conjunction with the accompanying drawings, in which:

FIG. 1 shows experimental results of inhibitory effects of eupatilin on osteoclast differentiation in microscopic images of osteoclastogenesis at various concentrations of eupatilin (A); a graph in which the numbers of TRP⁺ multinucleated osteoclasts (MNCs) are plotted against eupatilin concentrations (B); immunofluorescent images of F-actins in cells treated with eupatilin (C); and a graph in which the cytotoxicity of eupatilin is examined;

FIG. 2 shows graphs in which expression patterns of the osteoclast differentiation markers TRAP, OSCAR, Integrin av, DC-STAMP, integrin b3, and OC-STAMP are measured by real-time PCR;

FIG. 3 shows experimental data of effects of eupatilin on factors involved in the differentiation of mouse bone marrow cells (MBMC) into osteoclasts in a gel electrophoresis image for phosphorylated IκB, Akt and GSK3β (A); microscopic images for osteoclastogenesis and a graph in which the numbers of TRP⁺ osteoclasts (OC) are shown upon the retroviral overexpression of IKKβ (B); microscopic images for osteoclastogenesis and a graph in which the numbers of TRP⁺ osteoclasts (OC) are shown upon the overexpression of Akt (C); and a graph in which the activity of NF-κB is depicted;

FIG. 4 shows experimental data of effects of eupatilin upon the overexpression of GSK3β in microscopic images of TRP⁺ multinucleated osteoclasts (A); and a graph (B);

FIG. 5 shows experimental data of effects of eupatilin in human peripheral blood mononucleated cells (PBMC) in microscopic images and a graph illustrating the generation of TRP⁺ osteoclasts (A); and in a gel electrophoresis image illustrating the phosphorylation of osteoclast differentiation-related factors (B);

FIG. 6 shows experimental data of inhibitory effects of eupatilin on bone resorption in microscopic images illustrating the expression of F-actin (A); graphs illustrating Pit generation and bone resorption rates (B); and a graph illustrating the expression levels of Cathepsin K;

FIG. 7 shows experimental data of therapeutic effects of eupatilin in LPS-induced osteoporosis mouse models in CT images for bone density and histochemical images obtained by H&E and TRAP staining (A); and graphs illustrating the extents of osteoporosis on the CT images (B to E);

FIG. 8 shows experimental data of therapeutic effects of eupatilin in ovariectomized mice (OVX) models in CT images for bone density and histochemical images obtained by H&E and TRAP staining (A); and graphs illustrating the extents of osteoporosis on the CT images (B to E); and

FIG. 9 shows experimental data of effects of eupatilin on the differentiation of mesenchymal stem cells isolated from the mouse calvaria into osteoblasts.

### DESCRIPTION OF THE PREFERRED EMBODIMENTS

In accordance with an aspect thereof, the present invention provides a pharmaceutical composition comprising eupatilin as an active ingredient inhibitory of osteoclast differentiation.

In accordance with another aspect thereof, the present invention provides a pharmaceutical composition comprising eupatilin as an active ingredient inhibitory of the bone metastasis attributable to osteoclast differentiation.

In this regard, the pharmaceutical composition is preferably used to inhibit the bone metastasis of breast cancer cells or prostate cancer cells.

In accordance with another aspect thereof, the present invention provides a pharmaceutical composition comprising eupatilin as an active ingredient for inhibiting osteolysis.

In accordance with another aspect thereof, the present invention provides a pharmaceutical composition comprising eupatilin as an active ingredient for preventing and treating periodontitis.

Eupatilin, useful in the present invention, is represented by the following Chemical Formula 1. Eupatilin, found in Artemisia, can be obtained from Artemisia using typical extraction, isolation and purification methods.

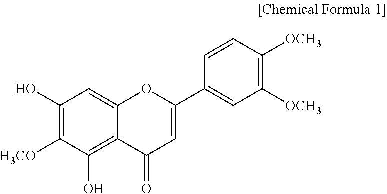 [SMILES: COc1ccc(-c2cc(=O)c3c(O)c(OC)c(O)cc3o2)cc1OC]

According to the present invention, eupatilin can effectively inhibit the differentiation of osteoclast progenitors into osteoclasts.

According to the present invention, eupatilin can effectively restrain the fusion of mononucleated osteoclasts into multinucleated osteoclasts.

According to the present invention, eupatilin can reduce the bone resorption activity of multinucleated osteoclasts. According to the present invention, eupatilin is inhibitory against the activity of NF-κB, a transcription factor involved in the transcription of various genes including, for example, immunoglobulins, interleukins, interleukin receptors, etc. in B cells, and against the activity of Akt and GSK3β, which are responsible for mitogenic activity. Therefore, eupatilin is evaluated to inhibit the expression of various factors involved in osteoclast differentiation. In this context, eupatilin is found to inhibit the phosphorylation of IκB, Akt, and GSK3β.

According to the present invention, eupatilin can reduce the intracellular level of NFAT-c1, a master transcription factor for osteoclast differentiation by inhibiting the expression of NFAT-c1 or by promoting the degradation of the protein. Hence, eupatilin is effective for inhibiting the differentiation and maturation of osteoclasts.

Fusion of mononucleated osteoclasts into multinucleated cells is implemented by an actin ring for the formation of which F-actin is necessary. According to the present invention, eupatilin can suppress the expression of F-actin essential to actin ring formation for RANK (receptor activator of NFκB)-induced osteoclast differentiation, and thus can effectively inhibit the fusion of mononucleated osteoclasts into multinucleated osteoclasts.

According to the present invention, eupatilin can suppress the expression of Cathepsin K, which is involved in bone resorption, and thus can effectively reduce the bone resorption activity of multinucleated osteoclasts.

According to the present invention, eupatilin can bring about an effective improvement in bone density in osteoporosis patients or postmenopausal women.

Thanks to the above-described effects of eupatilin, the pharmaceutical composition of the present invention can effectively suppress osteoclast differentiation.

Bone metastasis is induced by osteoclasts. Osteoclasts attract cancer cells, which results in bone metastasis. In this regard, a positive hormonal feedback loop between tumors and osteoclasts promotes tumor growth and deteriorates bone metastasis. Suppressive of such osteoclast differentiation, the pharmaceutical composition of the present invention can effectively inhibit metastasis. Particularly since most breast cancers and prostate cancers metastasize into bone, the pharmaceutical composition of the present invention is expected to exert more effective inhibitory activity against the metastasis of breast cancer and prostate cancer into bone.

The bone resorption of multinucleated osteoclasts is responsible for osteolysis. The pharmaceutical composition of the present invention can effectively inhibit such osteolysis because it can suppress osteoclasts differentiation and the fusion of mononucleated osteoclasts into multinucleated osteoclasts and can reduce the bone resorption of multinucleated osteoclasts.

Periodontitis is a set of inflammatory diseases caused by microorganisms. In an inflammatory condition, bone loss is progressively increased with the formation of osteoclasts. The pharmaceutical composition of the present invention is applicable to the effective prevention and treatment of osteolysis because it can suppress osteoclasts differentiation and the fusion of mononucleated osteoclasts into multinucleated osteoclasts and can reduce the bone resorption of multinucleated osteoclasts.

In addition to eupatilin, the pharmaceutical composition of the present invention may further comprise at least one functionally identical or similar, active ingredient.

The pharmaceutical composition may contain eupalitin in an amount of 0.1 to 90 parts by weight, based on 100 parts by weight of the composition.

The composition may be administered orally or parenterally. For parenteral administration, the composition may be injected intraperitoneally, intrarectally, subcutaneously, intravenously, intramuscularly, intracervically, intracerebraoventricularly, or intrathoracically. The pharmaceutical composition of the present invention may be formulated into typical dosage forms.

For preventing or treating the diseases caused by osteoclast differentiation, the pharmaceutical composition of the present invention may be used alone or in combination with surgery, hormone therapy, pharmacological therapy or biological response modulators.

Depending on various factors including the patient's weight, sex, heath state, and diet, the route and time of administration, excretion rates, and the severity of disease, the dose of the pharmaceutical composition in accordance with the present invention may vary. For a preferred effect, the effective amount of the pharmaceutical composition of the present invention may range in a daily dose from 0.0001 to 100 mg/kg, and more preferably from 0.001 to 10 mg/kg, but is not limited thereto. The active ingredient according to the present invention may be administered in a single dosage, or may be divided into multiple dosages per day.

In addition, the pharmaceutical composition of the present invention may be prepared into various parenteral dosage forms for use in clinical practice. In this context, the pharmaceutical composition of the present invention may be formulated in combination with a diluent or excipient such as a filler, a thickener, a binder, a humectant, a disintegrant, a surfactant, etc. Also, the pharmaceutical composition of the present invention may be in a parenteral dosage form such as sterile aqueous solutions, non-aqueous solvents, suspensions, emulsions, lyophilizates, suppositories, and the like. Propylene glycol, polyethylene glycol, vegetable oils such as olive oil, and injectable esters such as ethyl oleate may be suitable for the non-aqueous solvents and suspensions. The basic materials of suppositories include Witepsol, Macrogol, Tween 61, cacao butter, laurin butter, glycerol, and gelatin.

A better understanding of the present invention may be obtained through the following examples which are set forth to illustrate, but are not to be construed as limiting the present invention.

### Example 1

### Eupatilin as Novel Osteoclast Differentiation Inhibitor

1-1. Effect of Eupatilin on Osteoclast Differentiation, Maturation or Inhibition

Incubation with M-CSF (macrophage-colony stimulating factor) and RANK (receptor activator of NFκB) ligand (RANKL) induced mouse bone marrow cells (MBMC) to differentiate into osteoclasts during which eupatilin was used at various concentrations under monitoring.

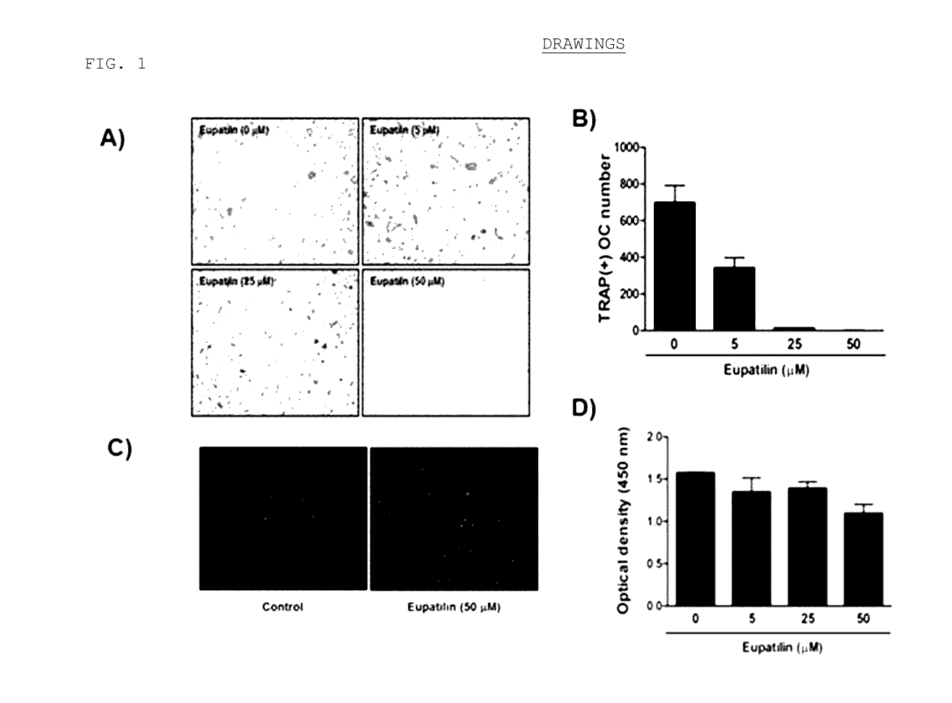

As shown in FIG. 1, 50 μM eupatilin completely suppressed osteoclast differentiation (FIG. 1A). In addition, as can be seen in FIG. 1B, eupatilin suppressed the formation of TRP⁺ multinucleated osteoclasts (MNCs) in a dose-dependent manner. Immunofluorescence detection of F-actin in MNCs demonstrated that eupatilin significantly reduced F-actin formation (FIG. 1C), but exerted no cytotoxicity at the concentration (FIG. 1D).

1-2. Change in Osteoclast Differentiation Marker Gene Levels with Eupatilin

During the differentiation of MBMC into osteoclasts in the presence of 50 μM eupatilin, the levels of osteoclast differentiation marker genes were measured by real-time PCR.

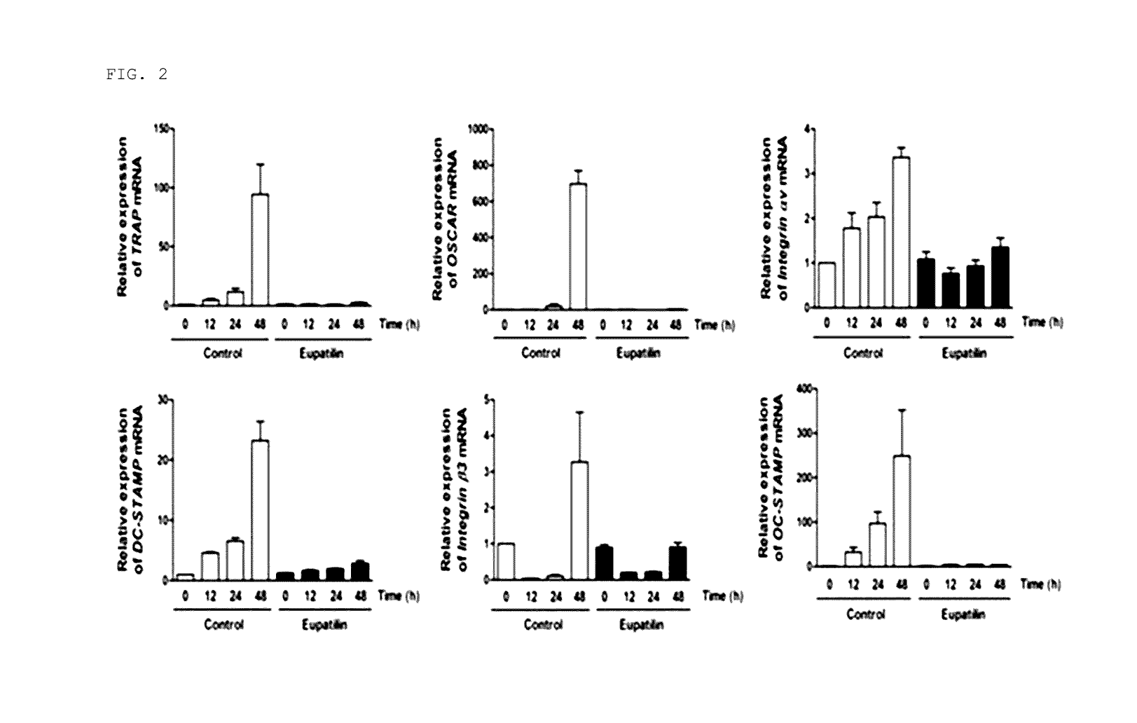

As shown in FIG. 2, eupatilin significantly reduced the levels of the markers TRAP, OSCAR, Integrin av, DC-STAMP, integrin b3, and OC-STAMP (FIG. 2).

1-3. Factors Relevant to Inhibitory Activity of Eupatilin Against Osteoclast Differentiation

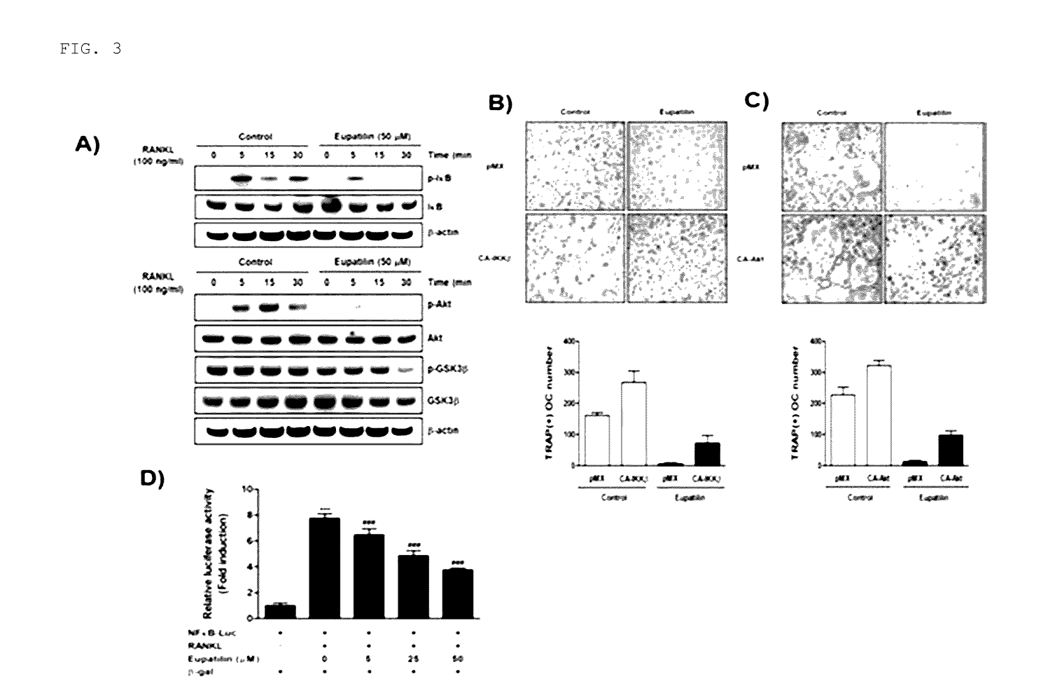

During the differentiation of MBMC into osteoclasts, phosphorylation levels of IkB, Akt, and GSK3b were significantly reduced in eupatilin-treated groups, compared to control groups (FIG. 3A). Retroviral overexpression of catalytically active IKKb or Akt induced partial formation of TRP⁺ osteoclasts (FIGS. 3B and 3C). An NF-kB-luciferase assay was conducted to examine whether the RANK-mediated NF-kB activation was modulated by eupatilin. As a result, eupatilin was found to inhibit RANK-mediated NF-kB activation (FIG. 3D).

1-4. Relationship Between Eupatilin and GSK3b

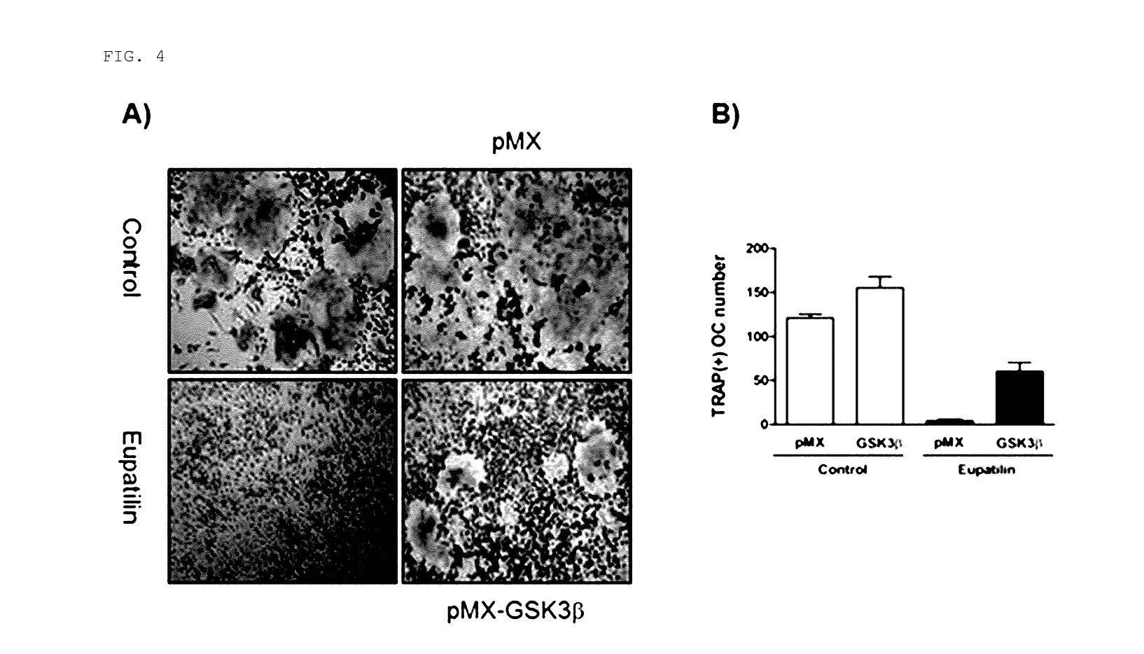

Since the phosphorylation of NF-kB p50-p65 dimer might influence osteoclast differentiation due to the reduction of phosphorylated GSK3b by eupatilin, an examination was made to see whether the overexpression of GSK3b suppressed the effect of eupatilin. Even in the presence of eupatilin, as shown in FIGS. 4A and 4B, a significant quantity of MNCs was observed due to the overexpression of GSK3b. The effect of eupatilin was found to be significantly further reduced by the overexpression of GSK3b than either Akt or IKKb, suggesting that eupatilin is likely to target the substrate of GSK3b or a proteome associated therewith.

1-5. Effect of Eupatilin in Human Peripheral Blood Mononucleated Cells (PBMC)

Effects of eupatilin on the differentiation of human PBMC into osteoclasts were observed.

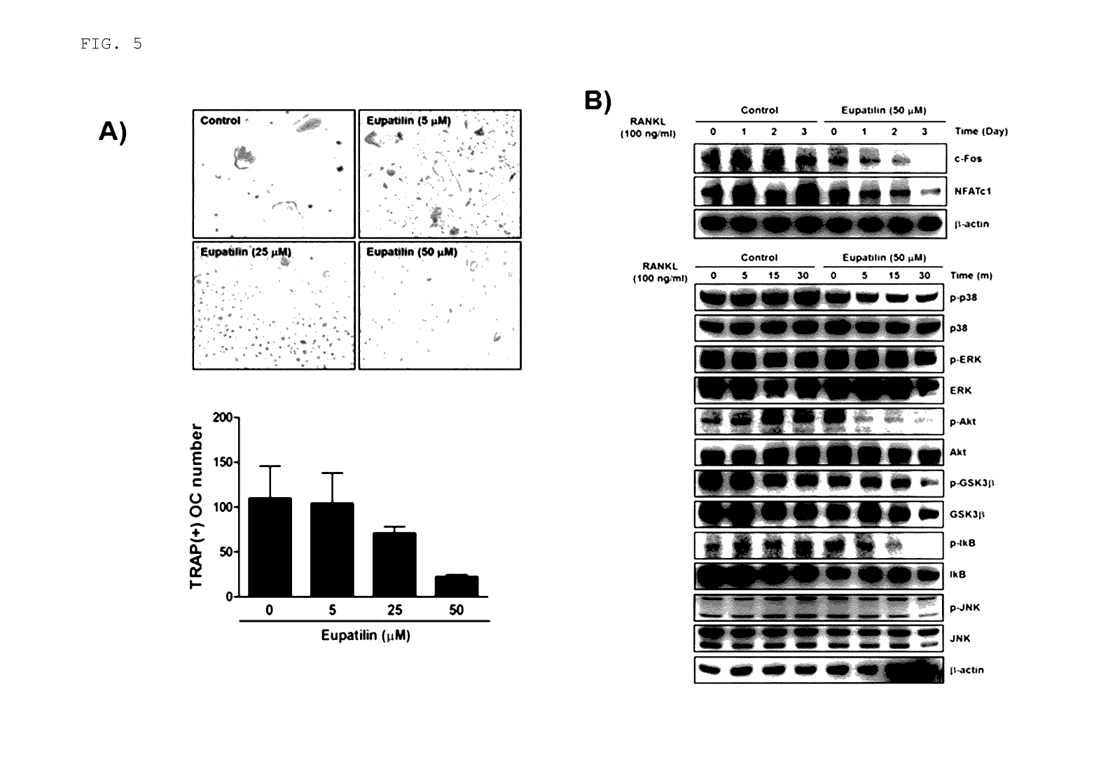

As can be seen in FIG. 5, eupatilin was also found to significantly reduce at a concentration of 50 μM the formation of TRP⁺ osteoclasts and the expression of c-Fos, NFATc1, p-Akt, p-GSK3b, and p-IkB.

With regard to the differentiation of MBMC and human PBMC into osteoclasts, eupatilin was found to inhibit the activity of NF-kB associated with inflammation signaling, the activity of Akt and GSK3b associated with mitogenic activity, and the expression of the transcription factor NFATc1 important for osteoclast differentiation, and to effectively suppress at a concentration of 50 μM the differentiation and maturation of osteoclasts.

1-6. Inhibition of Eupatilin Against RANKL-Mediated Bone Resorption

Effects of eupatilin on RANKL-mediated bone-resorption were measured using dentin.

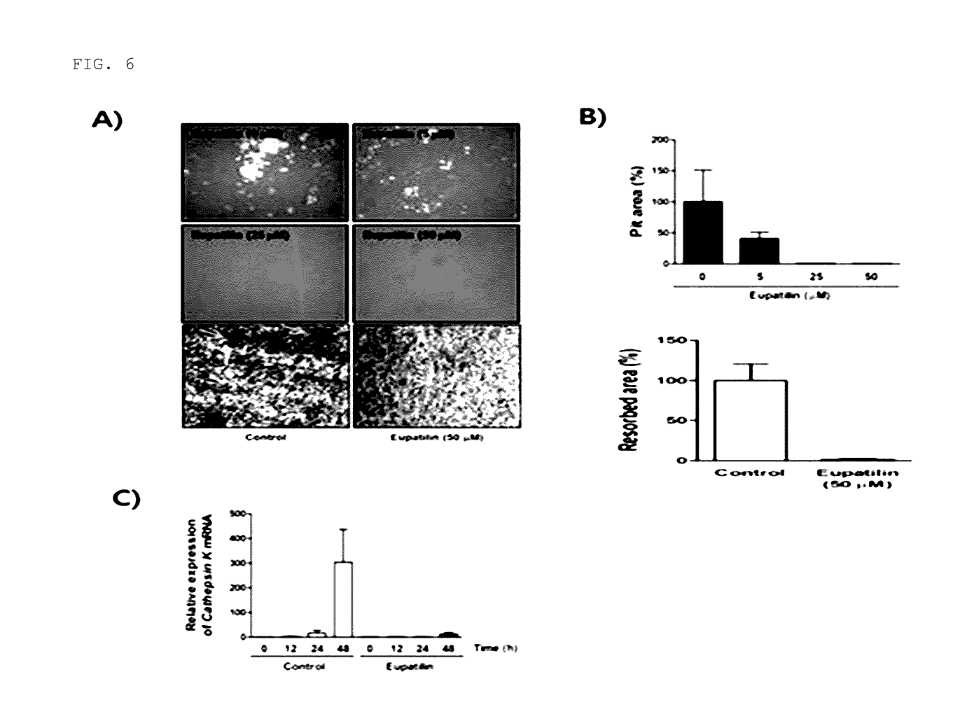

As shown in FIG. 6A, eupatilin remarkably suppressed the expression of F-actin for the formation of osteoclast actin rings differentiated by RANK signaling. Further, eupatilin reduced the expression of Pit (FIG. 6B), thereby significantly decreasing the expression of Cathepsin K responsible for bone resorption in a time-dependent manner.

1-7. Conclusion

Eupatilin is a potent osteoclast differentiation inhibitor in mice and humans, and greatly reduces the bone resorption of osteoclasts. Targeting NF-kB, which is involved in general inflammation singling, eupatilin may have influences on the formation of pro-inflammatory cytokines and W chemokines. Because eupatilin inhibits the formation of TRP⁺ MNCs by decreasing the absolute level of NFATc1 through the inhibition of NFATc1 expression or the promotion of NFATc1 degradation, the osteoclast inhibition mechanism of eupatilin was found to be associated with anti-inflammation signaling.

### Example 2

### Preventive and Therapeutic Effect of Eupatilin on Osteoporosis

2-1. Effect of Eupatilin in LPS-Induced Osteoporosis Model

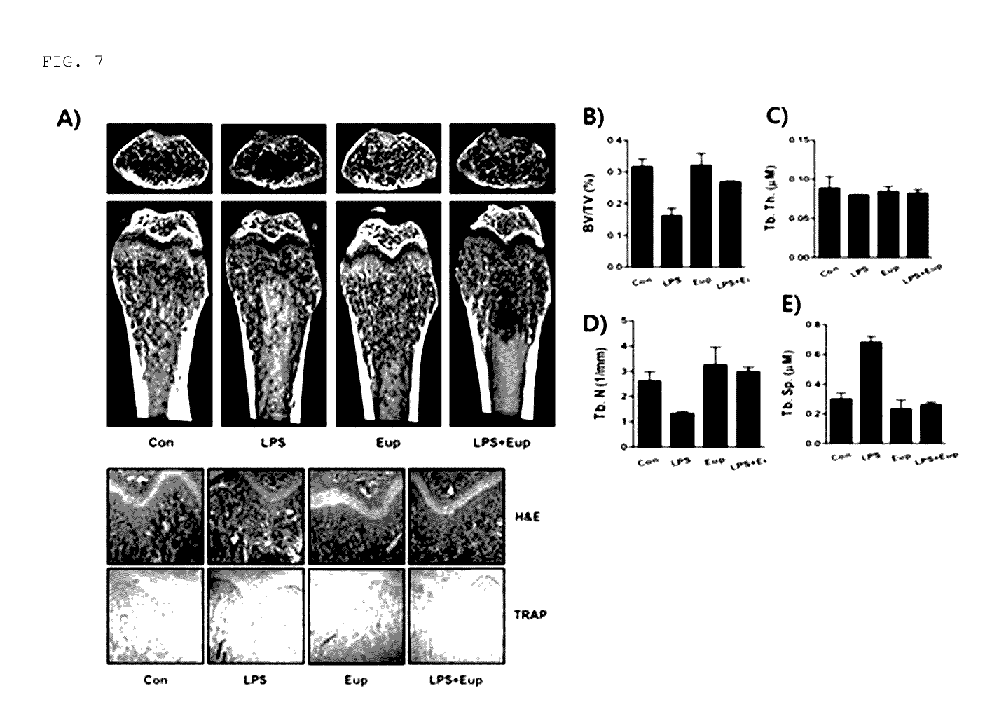

An LPS-induced osteoporosis model constructed by intraperitoneally injecting the potent inflammation factor LPS was monitored for bone density by CT after treatment with 50 μM eupatilin. The results are given in FIG. 7A. As shown, 50 μM eupatilin significantly reduced LPS-induced osteoporosis.

In addition, H&E and TRAP staining demonstrated the activity of eupatilin to promote osteanagenesis and inhibit osteoclast differentiation, as shown in the bottom panel of FIG. 7. In FIGS. 7B to 7E, eupatilin was observed to improve bone density upon treatment with LPS or LPS+ eupatilin.

2-2. Effect of Eupatilin in Ovariectomized Model

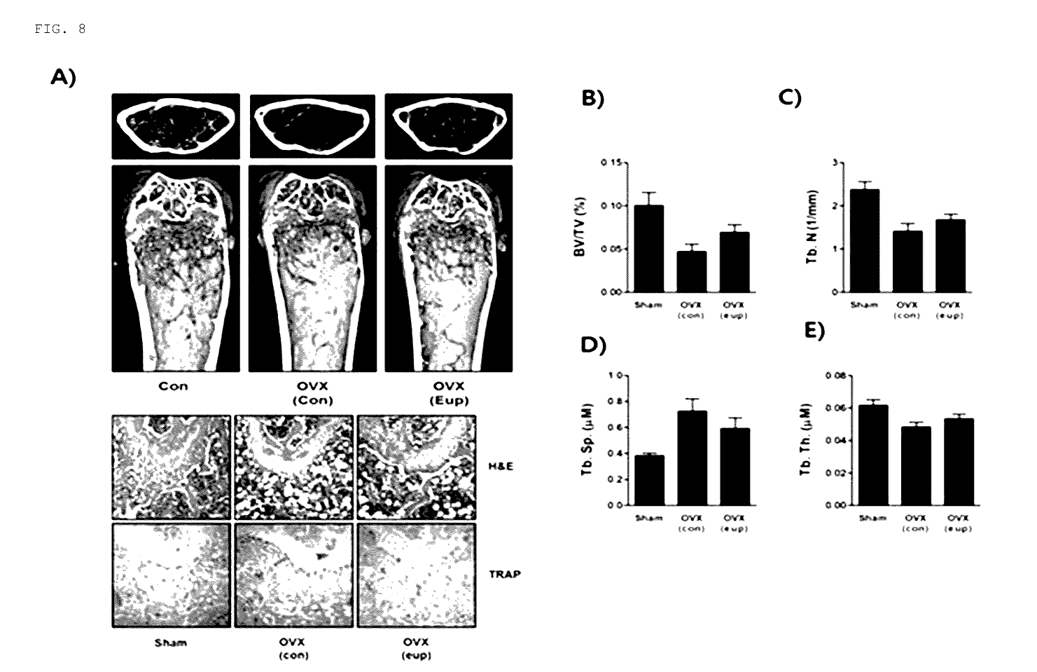

Ovariectomized mice (OVX) were monitored for menopausal osteoporosis by CT after treatment with 50 μM eupatilin. As shown in FIG. 8, eupatilin significantly reduced bone density loss.

2-3. Effect of Eupatilin on Osteoblast Differentiation

Osteoblasts differentiated from mesenchymal stem cells were measured for ALP activity in the presence of eupatilin.

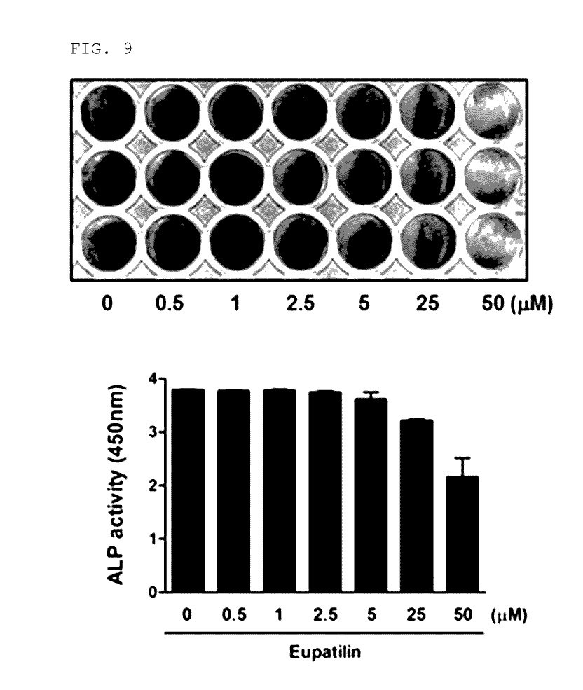

As shown in FIG. 9, eupatilin did not influence the activity of osteoblasts at up to 25 μM while 50 μM eupatilin reduced the activity of osteoblasts by 50%. Accordingly, eupatilin is evaluated to have preventive and therapeutic effects on osteoporosis by suppressing the differentiation of osteoclasts and the formation and bone resorption of multinucleated osteoclasts.

2-4. Conclusion

Eupatilin has potent inhibitory activity against osteoclast differentiation and bone resorption. This effect is based on the function of reducing the activity or expression level of NF-kB and NFATc1. The reduced phosphorylation of GSK3b and the recovery of MNCs upon the overexpression of GSK3b suggest the possibility that the target of eupatilin might be a protein associated with GSK3b. Eupatilin was also observed to effectively prevent osteoporosis in LPS-induced osteoporosis model mice and ovariectomized mice. Eupatilin seems to be not involved in W the activity of osteoblasts. Therefore, eupatilin can be used as a medicament preventive of osteoporosis and as an active ingredient for suppressing the symptoms attributed to inflammatory osteoclast differentiation, as in periodontitis.

As demonstrated by the present invention, eupatilin can effectively suppress the differentiation of osteoclasts progenitors into osteoclasts and the fusion of mononucleated osteoclasts into multinucleated osteoclasts, and reduce the bone resorption of multinucleated osteoclasts. Therefore, the pharmaceutical composition comprising eupatilin in accordance with the present invention can effectively suppress osteoclast differentiation, with the consequent inhibition of bone metastasis and osteolysis, and can be applied to the prophylaxis or therapy of periodontitis.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method of inhibiting osteoclast differentiation in a subject in need thereof, comprising:
providing a pharmaceutical composition comprising a therapeutically effective amount of eupatilin, wherein the eupatilin is the sole active ingredient for inhibiting the osteoclast differentiation; and
administering the pharmaceutical composition to the subject, wherein the osteoclast differentiation is inhibited.

2. A method of inhibiting osteoclast differentiation-induced bone metastasis in a subject in need thereof, comprising:
providing a pharmaceutical composition comprising a therapeutically effective amount of eupatilin, wherein the eupatilin is the sole active ingredient for inhibiting the osteoclast differentiation-induced bone metastasis; and
administering the pharmaceutical composition to the subject, wherein the osteoclast differentiation-induced bone metastasis is inhibited.

3. The method of claim 2, wherein the bone metastasis results from invasion of breast cancer cells or prostate cancer cells into bone.

4. A method of inhibiting osteolysis in a subject in need thereof, comprising:
providing a pharmaceutical composition comprising a therapeutically effective amount of eupatilin, wherein the eupatilin is the sole active ingredient for inhibiting the osteolysis; and
administering the pharmaceutical composition to the subject, wherein the osteolysis is inhibited.

5. A method of preventing or treating periodontitis in a subject in need thereof, comprising:
providing a pharmaceutical composition comprising a therapeutically effective amount of eupatilin, wherein the eupatilin is the sole active ingredient for inhibiting the periodontitis; and
administering the pharmaceutical composition to the subject, wherein the periodontitis is prevented or treated.
